(19) United States
(12) Patent Application Publication
(10) Pub. No.: US 2010/0160704 A1
(43) Pub. Date: Jun. 24, 2010

(54) ALKYLATION OF AROMATICS WITH HIGH ACTIVITY CATALYST

(76) Inventors: Robert Schmidt, Barrington, IL (US); Deng-Yang Jan, Elk Grove Village, IL (US); Robert James, Northbrook, IL (US)

Correspondence Address: HONEYWELL/UOP;PATENT SERVICES, 101 COLUMBIA DRIVE, P O BOX 2245 MAIL STOP AB/2B, MORRISTOWN, NJ, 07962, US

(21) Appl. No.: 12/339,887

(22) Filed: Dec. 19, 2008

(51) Int. Cl. C07C 2/12

(52) U.S. Cl. 585/468

(57) ABSTRACT

A process for the alkylation of aromatics such as benzene with olefins such as cumene and ethylbenzene and in a reaction zone containing high activity UZM-8 catalysts. The process is carried out at a high weight hourly space velocity (WHSV) while still achieving complete olefin conversion in the reaction zone.

### FIELD OF THE INVENTION

This invention relates to processes for producing alkylated aromatic compounds such as cumene and ethylbenzene by alkylation and/or transalkylation using high activity zeolite catalysts.

### BACKGROUND OF THE INVENTION

Alkylation of aromatic compounds with C₂ to C₇ olefins and transalkylation of polyalkylaromatic compounds are two common reactions for producing monoalkylated aromatic compounds. Examples of these two reactions that are practiced industrially to produce cumene (isopropylbenzene) are the alkylation of benzene with propylene and the transalkylation of benzene with a diisopropylbenzene (DIPB). The alkylation reaction forms cumene and common byproducts such as DIPBs and triisopropylbenzenes (TIPBs). DIPBs, TIPBs, and some of the higher polyisopropylbenzenes can be readily transalkylated by benzene to produce cumene. Combining alkylation and transalkylation in a process can thus maximize cumene and ethylbenzene production. Such processes can have two reactions zones, one for alkylation and the other for transalkylation, or a single reaction zone in which both alkylation and transalkylation occur. Single reaction zones may be desirable due to lower equipment costs.

Zeolite catalysts have been proposed and used for alkylating and transalkylating aromatics including Beta, UZM-8, Y, ZSM-5, PSH-3, MCM-22, MCM-36, MCM-49, and MCM-56. Regardless of whether the reaction is alkylation or transalkylation, it is of critical importance that the zeolitic catalyst exhibits not only the capability to initially perform its specified functions, but also that it has the capability to perform these functions satisfactorily for prolonged periods of time. The analytical terms used in the art to measure how well a particular catalyst performs its intended functions in a particular hydrocarbon reaction environment are activity, selectivity, and stability. “Activity” is a measure of the catalyst's ability to convert hydrocarbon reactants into products at a specified severity level, where severity level means the conditions used—that is, the temperature, pressure, contact time, concentration of reactants, and presence of diluents such as paraffins or contaminants such as water. “Selectivity” refers to the amount of desired product or products obtained relative to the amount of reactants charged or converted. “Stability” refers to the rate of change with time of the activity and selectivity parameters. A smaller stability rate suggests a more stable catalyst.

In a process for producing cumene, for example, activity commonly refers to the amount of conversion of propylene that takes place at a specified severity level and is typically measured by olefin content of the alkylation reactor effluent; selectivity refers to the amount of cumene yield, relative to the amount of the propylene consumed that is obtained at the particular activity or severity level; and stability is typically equated to the rate of change with time of activity, as measured by the position of the maximum temperature (due to the exothermic reaction) in the catalyst bed after a suitable interval of time.

Catalyst activity, selectivity and stability directly impact the rate at which alkylation and transalkylation reactions can be carried out. This rate is commonly measured as a weight hour space velocity (“WHSV”), which is calculated by dividing the weight flow rate of the component per hour by the zeolite weight. For example, commercial processes for producing cumene using a UZM-8 catalyst have been operated at an average of about 0.3 to 0.7 hr⁻¹ WHSV and a temperature range of about 110 to 160° C.

Although it would be desirable to run alkylation and transalkylation reactions at a significantly higher WHSV, commercial operations tend to be operated at a lower rate to prevent olefin breakthrough (i.e., less than complete olefin conversion through the catalyst bed) over the life of the catalyst bed.

### SUMMARY OF THE INVENTION

One embodiment is a process for producing cumene, in which a feed stream containing benzene is reacted with an alkylating agent including propylene in a reaction zone in the presence of a UZM-8 or UZM-8HS catalyst to produce an effluent including cumene. The process is carried out with a length to diameter ratio for each catalyst bed in the reaction zone of between about 1.5 and 3, a propylene WHSV of between about 2.1 hr⁻¹ and about 10.0 hr⁻¹, a reaction temperature of between about 60° and 160° C., and a molar ratio of benzene to propylene of between about 1 and about 3. The reaction results in complete olefin conversion.

Another embodiment is a process for producing ethylbenzene, in which a feed stream containing benzene is reacted with an alkylating agent including ethylene in a reaction zone in the presence of a UZM-8 or UZM-8HS catalyst to produce an effluent including ethylbenzene. The process is carried out with a length to diameter ratio for each catalyst bed in the reaction zone of between about 1.5 and 3, a WHSV of between about 1.5 hr⁻¹ and about 4.0 hr⁻¹, a reaction temperature of between about 150° and 280° C., and a molar ratio of benzene to ethylene of between about 1 and about 4. The reaction results in complete olefin conversion.

### BRIEF DESCRIPTION OF THE DRAWINGS

FIG. 1 is a chart illustrating percent conversion of propylene versus catalyst contact time according to Example 1.

FIG. 2 is a chart illustrating catalyst selectivity according to Example 1.

FIG. 3 is a chart illustrating the activity of regenerated catalyst according to Example 1.

FIGS. 4 and 5 are charts illustrating catalyst stability according to Example 1.

FIG. 6 is a chart illustrating percent conversion of ethylene versus catalyst contact time according to Example 2.

FIG. 7 is a chart illustrating percent ethylene conversion versus catalyst bed depth according to Example 2.

### DETAILED DESCRIPTION

The present invention relates to processes for the alkylation of aromatics with an alkylating agent in the presence of a UZM-8 catalyst. Embodiments of the present invention are directed to processes for producing cumene and ethylbenzene.

Although benzene is the principal feed aromatic of interest, feed aromatics such as alkyl-substituted benzenes, condensed ring systems generally, and alkylated derivatives thereof may be used. Examples of such feed aromatics are toluene, ethylbenzene, propylbenzene, diisopropylbenzene and so forth; xylene, mesitylene, methylethylbenzene, and so on; naphthalene, anthracene, phenanthrene, methylnaphthalene, dimethyl-naphthalene, and tetralin. Any alkyl groups present as substituent groups on the feed aromatic typically contain from 1 to 8 carbon atoms per group, more particularly from 1 to 4 carbon atoms per group. More than one feed aromatic can be used. The feed aromatic may be introduced into an alkylation catalyst bed in one or more aromatic feed streams. Each aromatic feed stream may contain one or more feed aromatics. Besides the feed aromatic(s), an aromatic feed stream may contain non-aromatics, including but not limited to saturated and unsaturated cyclic hydrocarbons that have the same, one more, or one less, number of carbon atoms as the feed aromatic. For example, an aromatic feed stream containing benzene may also contain cyclopentane, cyclohexane, cycloheptane, cyclopentenes, cyclohexenes, or cycloheptenes, as well as methylated versions of any of these hydrocarbons, or mixtures thereof. The feed stream may also contain contaminants including water, oxygenates including organic oxygenates such as alcohols, aldehydes and ketones, carbon monoxide and carbon dioxide.

The concentration of each feed aromatic in each aromatic feed stream may range from 0.01 to 100 wt %. Sources of benzene, toluene, xylene, and/or other feed aromatics include product streams from naphtha reforming units, aromatic extraction units, and petrochemical complexes for the producing para-xylene and other aromatics.

Feed olefins containing from 2 to 6 carbon atoms are the principal alkylating agents contemplated for the process disclosed herein. Examples of such feed olefins include C₂ to C₄ olefins, namely ethylene, propylene, butene-1, cis-butene-2, trans-butene-2, and isobutene. However, feed olefins having from 2 to 20 carbon atoms may be used effectively in the process disclosed herein. More than one feed olefin may be used. The feed olefin may be introduced into an alkylation catalyst bed in one or more olefinic feed streams. Each olefinic feed stream may contain one or more feed olefins. In addition to the feed olefin(s), an olefinic feed stream may contain non-olefins, such as paraffins that have the same number of carbon atoms as the olefin. For example, a propylene-containing olefinic feed stream may also contain propane, while an olefinic feed stream containing ethylene may also contain ethane and methane. The concentration of each feed olefin in each olefinic feed stream may range from 0.01 to 100 wt %. Sources of olefinic feed streams containing mixtures of olefins include refinery FCC propane/propylene streams, FCC ethane/ethylene/methane streams, naphtha cracking unit off gases, gas plant off gases, and other refinery streams.

The concentration of olefin in the feed stream may depend, in part, on the desired end product. For cumene alkylation, the propylene concentration may range from 50 wt % propylene for use in lower value end products to 99.8% for higher value products. For ethylbenzene alkylation, the ethylene concentration may range from 10 wt % ethylene to 99.8% ethylene.

In one embodiment, a transalkylation agent is also used. In theory, the transalkylation agent, if present, may be any compound that is capable of transalkylating with the alkylation substrate (e.g., benzene), mixing with the alkylating agent (e.g., ethylene), and decreasing the concentration of the alkylating agent at and downstream of the alkylation agent injection point. The transalkylation agent may have a number of characteristics that are consistent with the process objective of producing high yields of high-purity product ethylbenzene. First, the transalkylation agent should increase the alkylated aromatic yield by transalkylation, in addition to increasing yield by minimizing 1,1-DPE formation. In an ethylbenzene process, a polyethylbenzene, such as diethylbenzene, triethylbenzene, and so forth up to even hexaethylbenzene, is preferred because each can transalkylate to ethylbenzene, regardless of whether each is alkylated by ethylene. Because of the possibility of alkylation of the polyethylbenzene by ethylene, however, the lighter polyethylbenzenes are more preferred over the heavier polyethylbenzenes, with diethylbenzene being most preferred. More generally when alkylating a feed aromatic with a C₂-C₄ olefin, the transalkylation agent is an alkylated derivative of the feed aromatic having from one to six more C₂-C₄ alkyl groups than the feed aromatic.

A second characteristic of the transalkylation agent is that the transalkylation agent preferably decreases the molar ratio of aryl groups per alkyl groups in the alkylation reaction zone. This is usually not a limiting characteristic, however, because if the transalkylation agent has at least one aryl group and one alkyl group, then the transalkylation agent will decrease the molar ratio of aryl groups per alkyl group if the ratio is greater than 1. Transalkylation agents with two or more alkyl groups per aryl group will decrease the molar ratio of aryl groups per alkyl group if the ratio is greater than 0.5, and so on for transalkylation agents with more alkyl groups per aryl group. Third, the transalkylation agent preferably should not adversely affect the yield of the desired monoalkylated aromatic. For example in the context of ethylbenzene production, toluene and cumene are not preferred, because ethylene can alkylate toluene or cumene and produce byproducts that cannot be converted readily to ethylbenzene by alkylation or transalkylation. Even though generally present in the alkylation effluent, ethylbenzene is also not preferred, because ethylbenzene can shift the equilibrium of the reactions away from the formation of ethylbenzene and because ethylbenzene can react with ethylene to produce styrene and ultimately 1,1-DPE. Thus, it would be preferred to not recycle to the alkylation reaction zone a stream containing more than 75 wt % of the desired monoalkylated aromatic, such as the ethylbenzene or cumene product stream produced by the ethylbenzene or cumene column of the product separation zone. Fourth, the transalkylation agent preferably should not adversely affect the purity of the product stream containing the desired monoalkylated aromatic. For example in the context of ethylbenzene production, xylenes are not preferred because they are relatively difficult to separate from ethylbenzene by distillation. Another reason that xylenes are not preferred is that they can adversely affect ethylbenzene yield by alkylating with ethylene.

In general, the transalkylation agent, when present, is preferably a compound that corresponds to the alkylation substrate alkylated with at least one more alkyl group corresponding to the alkylation agent than the number of alkyl groups on the desired product of alkylating the alkylation substrate with the alkylating agent. In the general case, the transalkylation agent, when present, is different from the desired product of alkylating the alkylation substrate with the alkylation agent. Where the aromatic is benzene and the olefin is ethylene, the transalkylation agent can generally be a polyethylbenzene, and suitable transalkylation agents include di-, tri-, and tetra-ethyl aromatic hydrocarbons such as diethylbenzene, triethylbenzene, diethylmethylbenzene, diethylpropylbenzene, etc. Diethylbenzenes are preferred. Where the aromatic is benzene and the olefin is propylene, the transalkylation agent can generally be a polypropylbenzene, and suitable transalkylation agents include di-, tri-, and tetra-propyl aromatic hydrocarbons such as diisopropylbenzene, triisopropylbenzene, diisopropylmethylbenzene, triisopropylmethylbenzene, etc. Diisopropylbenzenes are especially preferred transalkylation agents.

The basic configuration of a catalytic aromatic alkylation process is known in the art and an example of such a process and system is in U.S. Pat. No. 7,268,267, which is incorporated herein by reference. The feed aromatic and the feed olefin are preheated and charged to an alkylation zone containing generally from one to four reactors in series. Suitable cooling means may be provided between reactors to compensate for the net exothermic heat of reaction in each of the reactors. Suitable means may be provided upstream of or with each reactor to charge additional feed aromatic, feed olefin, or other streams (e.g., effluent of a reactor, or a transalkylation stream containing one or more polyalkylbenzenes) to any reactor in the alkylation zone. Each alkylation reactor may contain one or more alkylation catalyst beds. Vessels or enclosures that can function as suitable reactors are known in the art. Common configurations of an alkylation zone include: one reactor with four catalyst beds; two reactors, each of which has two catalyst beds; and one reactor with four catalyst beds and a second reactor with two catalyst beds. The number of reactors is generally less than eight, and the number of catalyst beds in a given reactor is generally less than six. In one embodiment, the present invention utilizes a single reactor.

In the case of a single reactor operation, an exemplary number of beds is four with the catalyst distribution arranged in an ascending profile of 20%, 24%, 27%, and 29%, respectively, of total catalyst distributed in each bed. The advantage of an ascending catalyst distribution is to provide a greater amount of catalyst in the lag beds as a precaution to prevent any olefin from breaking through to the product in the event of upsets or feed contamination of the catalyst in the lead beds. The bed depth for each bed may range from about 1.5 meters to about 3 meters to ensure that complete olefin conversion occurs in each bed. A bed geometry of about 1.5 to 3 L/D ratio (L/D=bed length to bed diameter ratio) may be employed to ensure acceptable olefin feed distribution in each bed to avoid inadvertent olefin feed by-passing of catalyst contained in each of the catalyst beds. Such a single reactor, multi-bed design ensures that uniform usage of the catalyst occurs over the life of the catalyst which is expected to be greater than 5 years.

In the context of aromatic alkylation, the principal reaction that occurs in the alkylation zone is the alkylation of the feed aromatic by the feed olefin to produce a monoalkylated aromatic. In addition, other side reactions can also occur. For example, the feed aromatic can transalkylate with a polyalkylated aromatic to produce the monoalkylated aromatic. Also, the polyalkylated aromatic can be alkylated with the feed olefin. The effluent stream of each reaction zone thus may contain monoalkylated aromatic, polyalkylated aromatic that was either charged to the alkylation zone or was a byproduct of a side reaction, feed aromatic that was either charged to the alkylation zone or is the byproduct of a side reaction, feed olefin at low concentration, and C₁ to C₃ paraffins.

The catalysts are contained in a fixed-bed system or a moving-bed system with associated continuous catalyst regeneration whereby catalyst may be continuously withdrawn, regenerated and returned to the reactors. These alternatives are associated with catalyst-regeneration options known to those of ordinary skill in the art, such as: (1) a semi-regenerative unit containing fixed-bed reactors maintains operating severity by increasing temperature, eventually shutting the unit down for catalyst regeneration and reactivation; (2) a swing-reactor unit, in which individual fixed-bed reactors are serially isolated by manifolding arrangements as the catalysts become deactivated and the catalyst in the isolated reactor is regenerated and reactivated while the other reactors remain on-stream; (3) continuous regeneration of catalyst withdrawn from a moving-bed reactor, with reactivation and return to the reactors of the reactivated catalyst as described herein; or: (4) a hybrid system with semi-regenerative and continuous-regeneration provisions in the same zone. In one embodiment of a fixed-bed semi-regenerative system, a fixed-bed UZM-8 alkylation zone is added to an existing fixed-bed semi-regenerative Beta zeolite alkylation process unit to process streams upstream of the Beta zeolite alkylation zone and enhance the yield, activity-stability, and/or yield stability obtained in the semi-regenerative system.

The catalyst used in the first of the sequential alkylation zones of the process disclosed herein contains one or more members of the family of aluminosilicate and substituted aluminosilicate zeolites designated UZM-8 and UZM-8HS. U.S. Pat. No. 7,091,390, incorporated herein by reference, describes the UZM-8 and UZM-8HS catalysts and their preparation in detail. UZM-8 zeolites are prepared in an alkali-free reaction medium in which only one or more organoammonium species are used as structure directing agents. In this case, the microporous crystalline zeolite (UZM-8) has a composition in the as-synthesized form and on an anhydrous basis expressed by the empirical formula:

Rᵣ^{p+}Al₁₋ₓEₓSi_{y}O_{z}

where R can be at least one organoammonium cation selected from protonated amines, protonated diamines, quaternary ammonium ions, diquaternary ammonium ions, protonated alkanolamines and quaternized alkanolammonium ions. A preferred organoammonium cation is one that is non-cyclic or does not contain a cyclic group as one substituent. Especially preferred may be an organoammonium cation containing at least two methyl groups as substituents. An example of a preferred cation may include diethyldimethylammonium (DEDMA), ethyltromethylammonium (ETMA), hexomethonium (HM), or a mixture thereof. The ratio of R to (Al+E) may be represented by “r” which can vary from about 0.05 to about 5. The value of “p”, which may be the weighted average valence of R, can vary from about 1 to about 2. The ratio of Si to (Al+E) as represented by “y” can vary from about 6.5-about 35. E can be an element, which may be tetrahedrally coordinated, can be present in the framework, and can be gallium, iron, chromium, indium or boron. The mole fraction of E may be represented by “x” and can have a value from 0-about 0.5, while “z” is the mole ratio of O to (Al+E) and can be given by the equation:

z=(r·p+3+4·y)/2

The UZM-8 zeolites can be prepared using both organoammonium cations and alkali and/or alkaline earth cations as structure directing agents. As in the alkali-free case above, the same organoammonium cations can be used here. Alkali or alkaline earth cations are observed to speed up the crystallization of UZM-8, often when present in amounts less than 0.05 M⁻/Si. For the alkali and/or alkaline earth metal containing systems, the microporous crystalline zeolite (UZM-8) has a composition in the as-synthesized form and on an anhydrous basis expressed by the empirical formula:

Mₘⁿ⁺Rᵣ^{p+}Al₁₋ₓEₓSi_{y}O_{z}

where M is at least one exchangeable cation and is selected from the group consisting of alkali and alkaline earth metals. Specific examples of the M cations include but are not limited to lithium, sodium, potassium, rubidium, cesium, calcium, strontium, barium and mixtures thereof. Preferred R cations include without limitation DEDMA, ETMA, HM and mixtures thereof. The value of “m” which is the ratio of M to (Al+E) varies from about 0.01 to about 2. The value of “n” which is the weighted average valence of M varies from about 1 to about 2. The ratio of R to (Al+E) is represented by “r” which varies from 0.05 to about 5. The value of “p” which is the weighted average valence of R varies from about 1 to about 2. The ratio of Si to (Al+E) is represented by “y” which varies from about 6.5 to about 35. E is an element which is tetrahedrally coordinated, is present in the framework and is selected from the group consisting of gallium, iron, chromium, indium and boron. The mole fraction of E is represented by “x” and has a value from 0 to about 0.5, while “z” is the mole ratio of 0 to (Al+E) and is given by the equation

z=(m·n+r·p+3+4·y)/2

where M is only one metal, then the weighted average valence is the valence of that one metal, i.e. +1 or +2. However, when more than one M metal is present, the total amount of

Mₘⁿ⁺−Mₘ₁⁽ⁿ¹⁾⁺+Mₘ₂⁽ⁿ²⁾⁺+Mₘ₃⁽ⁿ³⁾⁺+ . . .

and the weighted average valence “n” is given by the equation:

$$\frac{{m}_{1} \cdot {n}_{1} + {m}_{2} \cdot {n}_{2} + {m}_{3} \cdot {n}_{3} + …}{{m}_{1} + {m}_{2} \cdot + {m}_{3} …}$$

Similarly when only one R organic cation is present, the weighted average valence is the valence of the single R cation, i.e., +1 or +2. When more than one R cation is present, the total amount of R is given by the equation:

Rᵣ^{P+}−Rᵣ₁^{(p1)+}+M_{Rr2}^{(p2)+}+Rᵣ₃^{(p3)+}

and the weighted average valence “p” is given by the equation:

$$\frac{{p}_{1} \cdot {r}_{1} + {p}_{2} \cdot {r}_{2} + {p}_{3} \cdot {r}_{3} + …}{{r}_{1} + {r}_{2} \cdot + {r}_{3} …}$$

The microporous crystalline zeolites used in the alkylation zone are prepared by a hydrothermal crystallization of a reaction mixture prepared by combining reactive sources of R, aluminum, silicon and optionally M and E. The sources of aluminum include but are not limited to aluminum alkoxides, precipitated aluminas, aluminum metal, sodium aluminate, organoammonium aluminates, aluminum salts and alumina sols. Specific examples of aluminum alkoxides include, but are not limited to aluminum ortho sec-butoxide and aluminum ortho isopropoxide. Sources of silica include but are not limited to tetraethylorthosilicate, colloidal silica, precipitated silica, alkali silicates and organoammonium silicates. A special reagent consisting of an organoammonium aluminosilicate solution can also serve as the simultaneous source of Al, Si, and R. Sources of the E elements include but are not limited to alkali borates, boric acid, precipitated gallium oxyhydroxide, gallium sulfate, ferric sulfate, ferric chloride, chromium nitrate and indium chloride. Sources of the M metals include the halide salts, nitrate salts, acetate salts, and hydroxides of the respective alkali or alkaline earth metals. R can be introduced as an organoammonium cation or an amine. When R is a quaternary ammonium cation or a quaternized alkanolammonium cation, the sources include but are not limited the hydroxide, chloride, bromide, iodide and fluoride compounds. Specific examples include without limitation diethyldimethylammonium (DEDMA) hydroxide, ethyltrimethylammonium (ETMA) hydroxide, tetramethylammonium hydroxide, tetraethylammonium hydroxide, hexamethonium bromide, tetrapropylammonium hydroxide, methyltriethylammonium hydroxide, DEDMA chloride, tetramethylammonium chloride and choline chloride. R may also be introduced as an amine, diamine, or alkanolamine that subsequently hydrolyzes to form an organoammonium cation. Specific non-limiting examples are N,N,N′,N′-tetramethyl-1,6-hexanediamine, triethylamine, and triethanolamine. Preferred sources of R without limitation are ETMAOH, DEDMAOH, and hexamethonium dihydroxide (HM(OH)₂).

The reaction mixture containing reactive sources of the desired components can be described in terms of molar ratios of the oxides by the formula:

aM_{2/nO}:bR_{2/p}O:1−cAl₂O₃:cE₂O₃:dSiO₂:eH₂O

where “a” varies from 0 to about 25, “b” varies from about 1.5 to about 80, “c” varies from 0 to 1.0, “d” varies from about 10 to about 100, and “e” varies from about 100 to about 15000. If alkoxides are used, it is preferred to include a distillation or evaporative step to remove the alcohol hydrolysis products. The reaction mixture is now reacted at a temperature of about 85° C. to about 225° C. (185 to 437° F.) and preferably from about 125° C. to about 150° C. (257 to 302° F.) for a period of about 1 day to about 28 days and preferably for a time of about 4 days to about 14 days in a sealed reaction vessel under autogenous pressure. After crystallization is complete, the solid product is isolated from the heterogeneous mixture by means such as filtration or centrifugation, and then washed with deionized water and dried in air at ambient temperature up to about 100° C. (212° F.).

The UZM-8 aluminosilicate zeolite, which is obtained from the above-described process, is characterized by an x-ray diffraction pattern, having at least the d-spacings and relative intensities set forth in Table A below:

TABLE A
d-Spacings and Relative Intensities for as-synthesized UZM-8

| 2-θ | d(Å) | I/I₀ % |
|---|---|---|
| 6.40-6.90 | 13.80-12.80 | w-s |
| 6.95-7.42 | 12.70-11.90 | m-s |
| 8.33-9.11 | 10.60-9.70 | w-vs |
| 19.62-20.49 | 4.52-4.33 | m-vs |
| 21.93-22.84 | 4.05-3.89 | m-vs |
| 24.71-25.35 | 3.60-3.51 | w-m |
| 25.73-26.35 | 3.46-3.38 | m-vs |

The UZM-8 compositions are stable to at least 600° C. (1112° F.) (and usually at least 700° C. (1292° F.)). The characteristic diffraction lines associated with typical calcined UZM-8 samples are shown below in table B. The as-synthesized form of UZM-8 is expandable with organic cations, indicating a layered structure.

TABLE B
d-Spacings and Relative Intensity for Calcined UZM-8

| 2-θ | d(Å) | I/I₀ % |
|---|---|---|
| 4.05-4.60 | 21.80-19.19 | w-m |
| 7.00-7.55 | 12.62-11.70 | m-vs |
| 8.55-9.15 | 10.33-9.66 | w-vs |
| 12.55-13.15 | 7.05-6.73 | w |
| 14.30-14.90 | 6.19-5.94 | m-vs |
| 19.55-20.35 | 4.54-4.36 | w-m |
| 22.35-23.10 | 3.97-3.85 | m-vs |
| 24.95-25.85 | 3.57-3.44 | w-m |
| 25.95-26.75 | 3.43-3.33 | m-s |

An aspect of the UZM-8 synthesis that contributes to some of its unique properties is that it can be synthesized from a homogenous solution. In this chemistry, soluble aluminosilicate precursors condense during digestion to form extremely small crystallites that have a great deal of external surface area and short diffusion paths within the pores of the crystallites. This can affect both adsorption and catalytic properties of the material.

As-synthesized, the UZM-8 material will contain some of the charge balancing cations in its pores. In the case of syntheses from alkali or alkaline earth metal-containing reaction mixtures, some of these cations may be exchangeable cations that can be exchanged for other cations. In the case of organoammonium cations, they can be removed by heating under controlled conditions. In the cases where UZM-8 is prepared in an alkali-free system, the organoammonium cations are best removed by controlled calcination, thus generating the acid form of the zeolite without any intervening ion-exchange steps. The controlled calcination conditions include the calcination conditions described herein below for the composite catalyst, and it may sometimes be possible desirable to perform the controlled calcination of the zeolite after the zeolite has been combined with a binder. On the other hand, it may sometimes be possible to remove a portion of the organoammonium via ion exchange. In a special case of ion exchange, the ammonium form of UZM-8 may be generated via calcination of the organoammonium form of UZM-8 in an ammonia atmosphere.

The zeolite, with or without a binder, can be formed into various shapes such as pills, pellets, extrudates, spheres, etc. Preferred shapes are extrudates and spheres. Extrudates are prepared by conventional means which involves mixing of zeolite either before or after adding metallic components, with the binder and a suitable peptizing agent to form a homogeneous dough or thick paste having the correct moisture content to allow for the formation of extrudates with acceptable integrity to withstand direct calcination. The dough then is extruded through a die to give the shaped extrudate. A multitude of different extrudate shapes are possible, including, but not limited to, cylinders, cloverleaf, dumbbell and symmetrical and asymmetrical polylobates. It is also within the scope of this disclosure that the extrudates may be further shaped to any desired form, such as spheres, by any means known to the art.

Spheres can be prepared by the well known oil-drop method which is described in U.S. Pat. No. 2,620,314, which is hereby incorporated herein by reference. The method involves dropping a mixture of zeolite, and for example, alumina sol, and gelling agent into an oil bath maintained at elevated temperature. The droplets of the mixture remain in the oil bath until they set and form hydrogel spheres. The spheres are then continuously withdrawn from the oil bath and typically subjected to specific aging treatments in oil and an ammoniacal solution to further improve their physical characteristics. The resulting aged and gelled particles are then washed and dried at a relatively low temperature of about 50 to 200° C. (122 to 392° F.) and subjected to a calcination procedure at a temperature of about 450 to 700° C. (842 to 1292° F.) for a period of about 1 to about 20 hours. This treatment effects conversion of the hydrogel to the corresponding alumina matrix.

The catalyst composite is dried at a temperature of from about 100° to about 320° C. (212 to 608° F.) for a period of from about 2 to about 24 or more hours and, usually, calcined at a temperature of from 400° to about 650° C. (752 to 1202° F.) in an air atmosphere for a period of from about 1 to about 20 hours. The calcining in air may be preceded by heating the catalyst composite in nitrogen to the temperature range for calcination and holding the catalyst composite in that temperature range for from about 1 to about 10 hours.

The binder used in the catalyst composite preferably contains less alkali and alkaline earth metals than the UZM-8, and more preferably contains little or no alkali and alkaline earth metals. Therefore, the catalyst composite has a content of alkali and alkaline earth metals of less than that of the UZM-8 zeolite used in forming the catalyst composite, owing to the binder effectively lowering the alkali and alkaline earth metals content of the catalyst composite as a whole.

As indicated by the Examples below, certain UZM-8 catalysts may have particularly favorable activity characteristics for operating at a high WHSV. For example, UMZ-8 catalysts in which R is DEDMA or ETMA may be particularly suitable. More specifically, UZM-8 catalysts with a AL₂O₃:DEDMA or AL₂O₃:ETMA molar ratio of less than 6, more particularly less than 4, even more particularly of about 2 may have particularly high activity.

Alkylation conditions for the alkylation zone includes a molar ratio of aryl groups per alkyl group of generally from 25 to about 1. The molar ratio may be less than 1, and it is believed that the molar ratio may be 0.75 or lower. For cumene and benzene alkylation, the molar ratio is preferably at least 1.2 and at most 4. In general, for a given molar ratio of alkylation substrate per alkylation agent, especially an olefinic alkylation agent, the greater the molar ratio of aryl groups to alkyl group in the feed stream, the less is the rise in temperature in the alkylation zone that occurs as a result of the alkylation reactions. The alkylation reactions are considered to be moderately exothermic. Although an alkylation zone, an alkylation reactor, and/or an alkylation catalyst bed may have indirect heat exchange means to remove the heat as it is produced, each zone, reactor, or bed is preferably adiabatic, and so the outlet temperature of the effluent stream is higher than the inlet temperature of the reactants. An increase in R/FF, as well as an increase in the molar ratio of aryl groups to alkyl groups in the feed stream, increases the quantity of aryl groups available to act as a heat sink in the alkylation zone and thus decreases the temperature rise in the alkylation zone. While in practicing the process disclosed herein, the appropriate alkylation temperature may be generally from 60° C. (140° F.) to the critical temperature of the alkylation substrate, which may be 475° C. (887° F.) or even higher, the inlet temperature in the alkylation zone is generally from 60 to 280° C. (140 to 536° F.), and preferably from 100 to 250° C. (212 to 482° F.). For cumene alkylation the preferred inlet temperature is from about 60° to about 160° C. For ethylbenzene alkylation the preferred inlet temperature is from about 150° to about 250° C. Although the temperature rise that occurs in the alkylation zone could be from 10 to 190° C. (18 to 342° F.) depending on the total mass flows in the alkylation zone, the temperature rise is generally from 5 to 130° C. (9 to 234° F.), and preferably from 5 to 50° C. (9 to 90° F.).

As described previously, the temperature rise in the alkylation zone may be controlled by adjusting the molar ratio of aryl groups to alkyl group in the feed stream. Minimizing the temperature rise helps prevent high reactor outlet temperatures, which may cause undesirable side reactions, such as isomerization of aromatic side-chains leading to compounds such as n-propylbenzene and heavy aromatic formation, to occur. High alkylation temperatures can also cause vaporization of benzene and the desired monoalkylaromatic (e.g. ethylbenzene or cumene) in the alkylation zone. In one embodiment of the process disclosed herein, the temperature rise in the alkylation zone can be controlled by withdrawing an effluent stream from the alkylation reaction zone, optionally cooling a portion of the effluent stream, and recycling the cooled portion of the effluent stream to the reaction zone.

Alkylation is preferably performed in the liquid phase. Consequently, alkylation pressures preferably are sufficiently high to ensure at least a partial liquid phase. The pressure range for the reactions is usually from about 1379 to 6985 kPa(g) (200 to about 1000 psi(g)), more commonly from about 2069 to 4137 kPa(g) (300 to about 600 psi(g)), and even more commonly from about 3103 to 4137 kPa(g) (350 to about 600 psi(g)). Preferably, the alkylation conditions are sufficient to maintain benzene in a liquid phase and are supercritical conditions for propylene.

As previously discussed, the WHSV of the various feed components with respect to the zeolite, and particularly the WHSV of the olefin, is an important consideration for aromatic alkylation processes. Although a high oleinf WHSV is desirable because it signifies a more efficient use of the zeolite, many zeolite catalysts do not possess sufficient activity, selectivity and/or stability to operate at a WHSV of greater than 1 hr⁻¹ to produce an effluent with complete olefin conversion for a sufficient period of time to function viably in a commercial process. As used herein, “complete olefin conversion” means that, with respect to a particular reaction zone, the effluent includes less than about 20% olefins, and more particularly, less than about 10% olefins, even more particularly, less than about 5% olefins, and even more particularly less than about 1% olefins.

As further indicated in the examples set forth below, the UZM-8 catalysts of the present invention may be utilized in cumene alkylation at an olefin feed WHSV, with respect to the zeolite, of at least about 2.1 hr⁻¹, more particularly, between about 2.1 hr⁻¹ and about 10 hr⁻¹, even more particularly, between about 2.1 hr⁻¹ and about 4.0 hr⁻¹, and even more particularly between about 2.5 and about 3.5 hr⁻¹. The UZM-8 catalysts of the present invention may be utilized in ethylbenzene alkylation at an olefin feed WHSV of at least about 1.5 hr⁻¹, more particularly, between about 1.5 hr⁻¹ and about 4 hr⁻¹, and even more particularly, between about 1.5 hr⁻¹ and about 2.5 hr⁻¹. Increased WHSV can be accomplished by reducing the catalyst bed length and/or by increasing the flow of the alkylating agent.

The selection of a particular WHSV or WHSV range depends, in part, on the desired product. Many high value products require olefin conversion of greater than 95%, more particularly, greater than 99%. In such a case, the WHSV for propylene may be between about 2.1 hr⁻¹ and 4.0 hr⁻¹, more particularly, between about 2.5 hr⁻¹ and 3.5 hr⁻¹. The WHSV for ethylene may be between about 1.5 hr⁻¹ and 3.0 hr⁻¹, more particularly, between about 1.5 hr⁻¹ and 2.5 hr⁻¹. Lower value products that do not require such a high olefin conversion may be process at a significantly higher WHSV, up to about 10 hr⁻¹ for propylene and about 4 hr⁻¹ for ethylene. Similarly, the olefin concentration in the feed stream impacts WHSV in that high concentration olefin streams are generally utilized for high value end products, while lower concentration olefin streams are used for lower value end products.

The following examples further illustrate the processes disclosed herein.

### EXAMPLES

### Experimental Protocol

The catalysts set forth in Table 1 below were subjected to a series of experiments to determine performance for cumene alkylation and ethylbenzene alkylation.

Catalyst A was prepared using a commercially available Beta zeolite. The zeolite was first ammonium exchanged to lower sodium contents, extruded into a cylindrical extrudate of 1/16″ diameter, dried and then calcine to remove the organic template.

Catalyst B is a UZM-8 catalyst prepared using UZM-8 zeolite synthesized as follows. In a large beaker 85 grams of aluminum tri-sec-butoxide was added to 387 grams diethyldimethylammonium hydroxide (20%) with vigorous stirring. Next, 267 grams of Ultrasil (SiO₂), followed by 600 grams of de-ionized water was added. Then a solution containing 13.4 grams of NaOH dissolved in 48 grams of de-ionized water was added to the aluminosilicate solution with mixing. The resulting solution was stirred for 20 minutes and then the 140 grams of slurry seed was added to the gel and stirred for 20 minutes. The gel was then transferred to a 2-liter stirred reactor and heated to 150° C. in 2 hours, and crystallized for 216 hours. After digestion the material was filtered and washed with de-ionized water and dried at 50° C. XRD data showed a pure UZM-8 material. The elemental analysis showed Si=43.5%, Al=3.8%, Na=1.51%, LOI=21.1%, C=10.9%, H=3.2%, N=2.3%. Surface area data showed BET=517 sq. m/g, TPV=1.05 cc/g, mpv=0.135 cc/g.

As synthesized UZM-8 zeolite was ammonium ion exchanged using 1 gram ammonium nitrate and 10 grams of H₂O per gram of zeolite, filtered and then washed by deionized H₂O. This procedure was repeated three times and the resulting zeolite contained less than 250 ppm (by weight) of sodium on a volatile free basis. The ammonium exchanged UZM-8 was dried and then extruded into a pellet of cylindrical extrudate of 1/16″ diameter containing 70% zeolite and 30% alumina. The pellet was dried at 110° C., then activated at about 540° C. for 1 hour first in flowing N₂ and then in flowing air for 15 hours in a box oven. Thereafter, the oven was cooled down to 110° C.

Catalyst C is a UZM-8 catalyst prepared in the same manner as Catalyst B with the exception that UZM-8 was synthesized using liquid sodium aluminate instead of aluminum tri-sec-butoxide and the amount of DEDMA was reduced to about 2 DEDMA:Al2O3 molar ratio with the addition of UZM-8 slurry at less than 10% levels as seed. XRD data showed a pure UZM-8 material. The elemental analysis showed Si=42.5%, Al=3.9%, Na=1.62%, Si/Al=10.46, C=6.4%, H=4.3%, N=1.2%. Surface area data showed BET=517 sq. m/g, TPV=1.05 cc/g, mpv=0.135 cc/g. As synthesized UZM-8 zeolite was ammonium exchanged and then extruded into a catalyst of 70 wt % zeolite and 30 wt % Al₂O₃ following the same procedure described for the preparation of Catalyst B.

Catalyst D was prepared following the same procedure as the aforementioned procedure using UZM-8 zeolite synthesized as per the same synthesis procedure as that described in the preparation of Catalyst C.

Catalyst E is a UZM-8 catalyst prepared in the following manner. In a baffled makeup tank, 6 volatile free grams of UZM-8 (as dry seed) was added to 706 grams of de-ionized water and stirred at 600 RPM using a single turbine blade. In a separate beaker 38.3 grams of liquid sodium aluminate, 108.5 grams of ethyltrimethylammonium hydroxide and 4 grams of 50% NaOH solution were mixed, and then added to the makeup tank. Next, 137.2 grams of Ultrasil (SiO₂) was added in 5 minutes to the makeup tank followed by stirring for 20 minutes and then transferred to a 2-liter reactor. It was then heated in 2 hours to 150° C. and crystallized for 165 hours. The product was then centrifuged and washed with de-ionized water and dried at 50° C. XRD data shows a pure UZM-8 material. The Elemental Analysis data: Si=41.5%, Al=3.9%, Na=1.63%, Si/Al=10.22, C=6.7%, H=2.9%, N=1.5%. The Surface Area Data showed BET=505 sq. m/g, TPV=0.9 cc/g, mpv=0.134 cc/g. The zeolite was ammonium exchanged and made into a catalyst of 70 wt % zeolite and 30 wt % alumina as per the procedure described for Catalyst B.

Catalyst F was prepared following the same procedure as the aforementioned procedure using UZM-8 zeolite synthesized as per the same synthesis procedure as that described in the preparation of Catalyst E.

Catalyst G is a catalyst made using MCM-22 synthesized as per U.S. Pat. No. 4,954,325 and was formulated into a catalyst of 70 wt % zeolite and 30 wt % alumina following the same procedure described for Catalysts B-F for comparative purposes.

Catalyst H is a catalyst made using MCM-56 synthesized as per U.S. Pat. Nos. 5,362,697 and 5,827,491 and was formulated into a catalyst of 70 wt % zeolite and 30 wt % alumina following the same procedure described for Catalysts B-F for comparative purposes.

A summary of Catalysts A-H are set forth in Table 1 below.

TABLE 1

| | | UZM-8 | AL₂O₃ |
|---|---|---|---|
| Catalyst | Type | Template | mol. rat. |
| A | Beta | — | — |
| B | UZM-8 | DEDMA | 4 |
| C | UZM-8 | DEDMA | 2 |
| D | UZM-8 | DEDMA | 2 |
| E | UZM-8 | ETMA | 2 |
| F | UZM-8 | ETMA | 2 |
| G | MCM-22 | — | — |
| H | MCM-56 | — | — |

### Example 1

### Cumene Alkylation

Benzene and propylene of a molar ratio of 2.5 were introduced into a fixed bed reactor containing cylindrical extruded catalyst in 1/16″ diameter. A portion of the product effluent was recycled with an effluent to fresh feed ratio of 6.0 on a weight basis. A multiple point sampling device was installed so the product compositions including the unconverted propylene were monitored along the catalyst bed. The temperature profiles along the catalyst bed were also monitored to provide another mean to gauge the catalyst activity. The catalyst was loaded at a constant volume and the performance was plotted against zeolite contact time to differentiate performance of different zeolite on a constant zeolite weight basis.

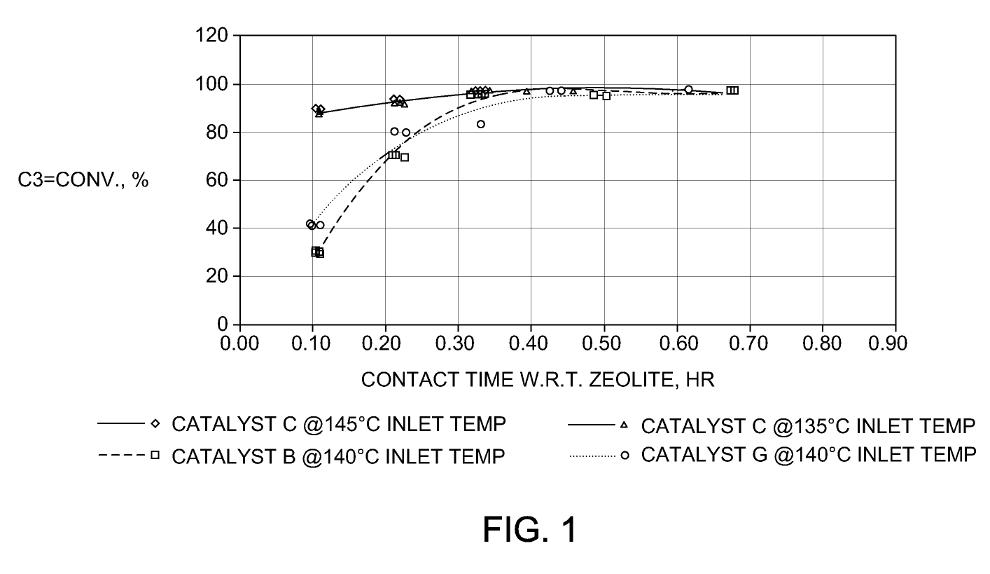

FIGS. 1-5 provide the results of Example 1. FIG. 1 is a chart showing the percent conversion of propylene versus contact time with respect to quantities up to the points where the product was sampled. FIG. 1 demonstrates that Catalyst C in particular achieved high olefin conversion at two different inlet temperatures, which achieved at least about 85% conversion within about 0.1 hours of contact time in a laboratory sized reactor. This indicates that Catalyst C could be used in a commercial process yielding lower value end products at an olefin WHSV (with respect to zeolite) of up to about 10 hr⁻¹ while still achieving complete olefin conversion. For higher value products, complete conversion (taking into account that it is difficult to achieve 100% conversion under laboratory conditions) occurred at about 0.33 hr. This indicates that Catalyst C could be used in a commercial operation at an olefin WHSV of between about 2.1 hr⁻¹ to about 4.0 hr⁻¹, more particularly between about 3.0 hr⁻¹ to about 4.0 hr⁻¹, while still achieving complete olefin conversion.

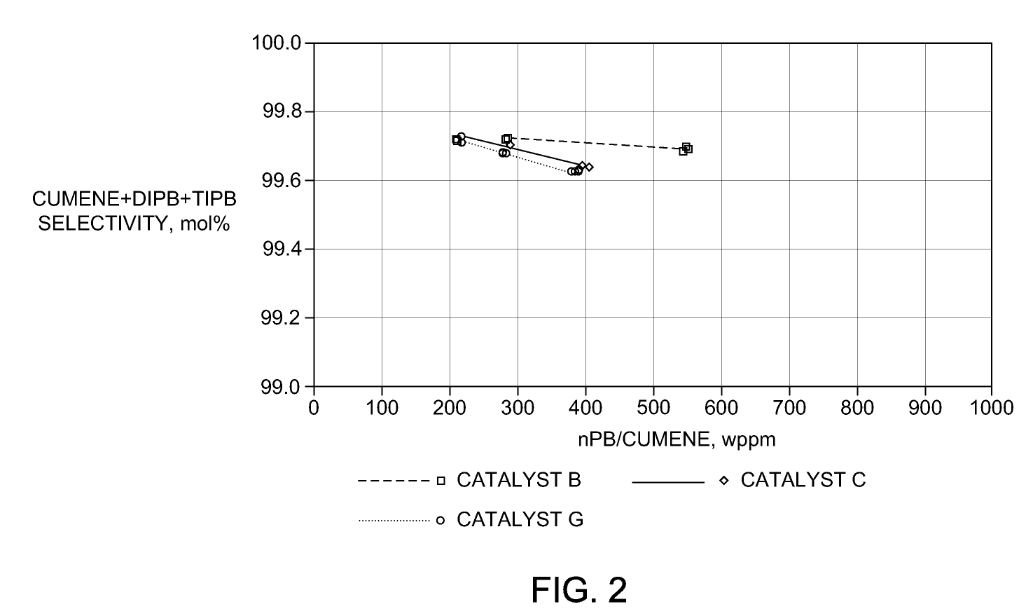

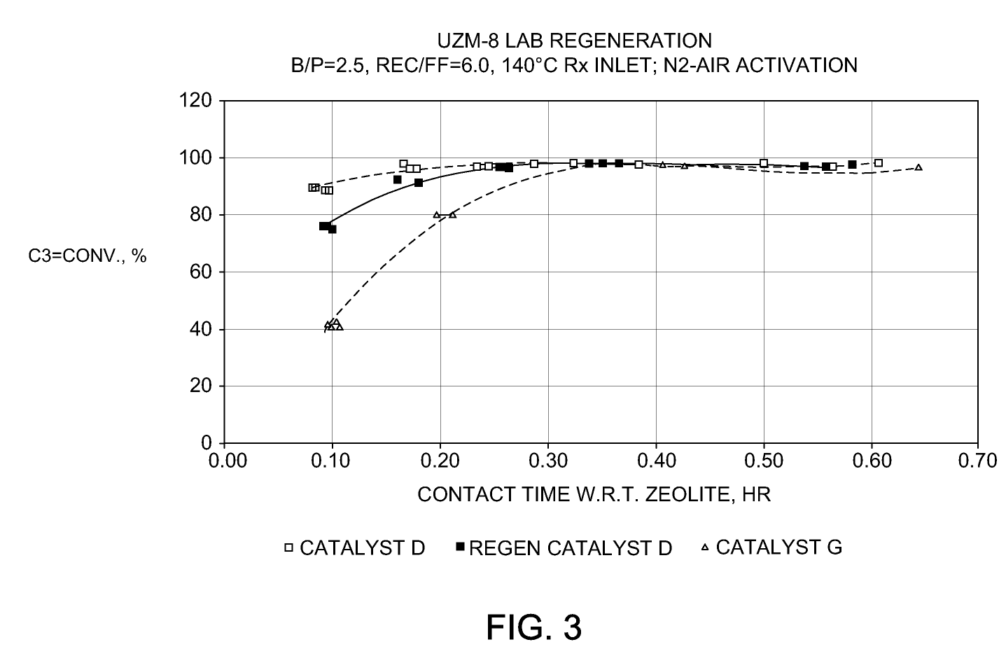

FIG. 2 shows catalyst selectivity for Catalysts B, C, and G, and demonstrates. Catalyst C has a higher total alkylate selectivity than Catalyst G. FIG. 3 is a chart illustrating the activity of fresh and regenerated forms of Catalyst D, which was synthesized using the same procedure as UZM-8 formulated into Catalyst C. The activity of the regenerated UZM-8 Catalyst D was only slightly lower than the fresh Catalyst G made using MCM-22.

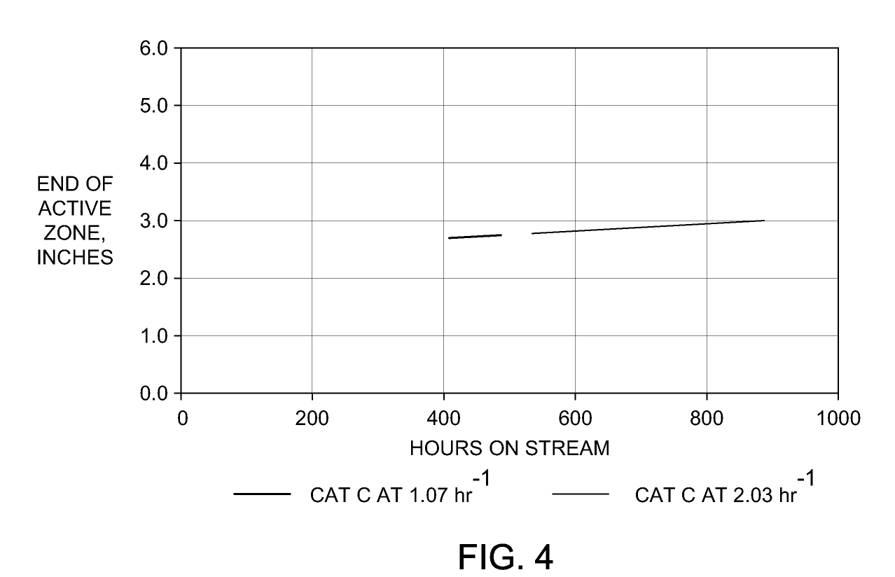

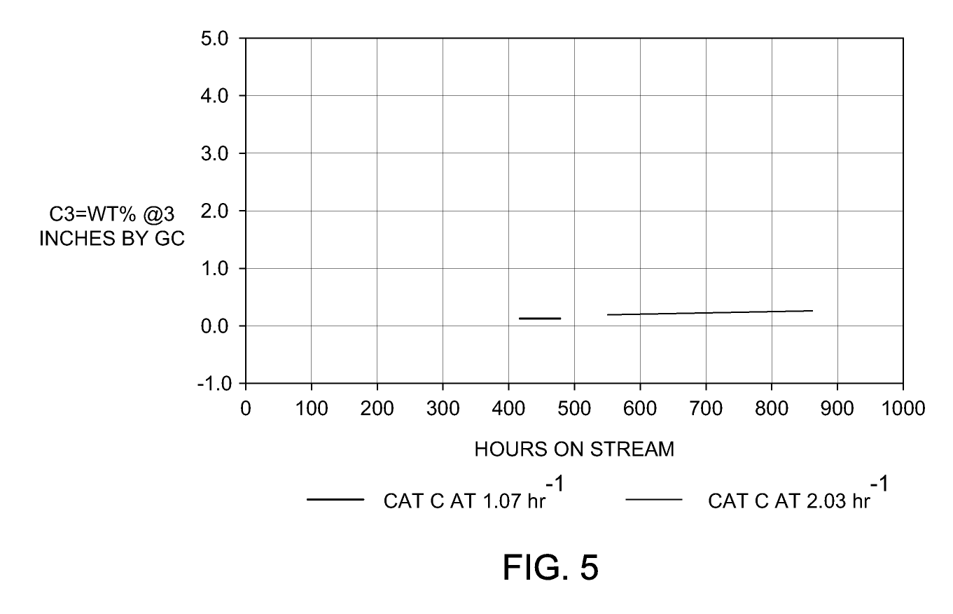

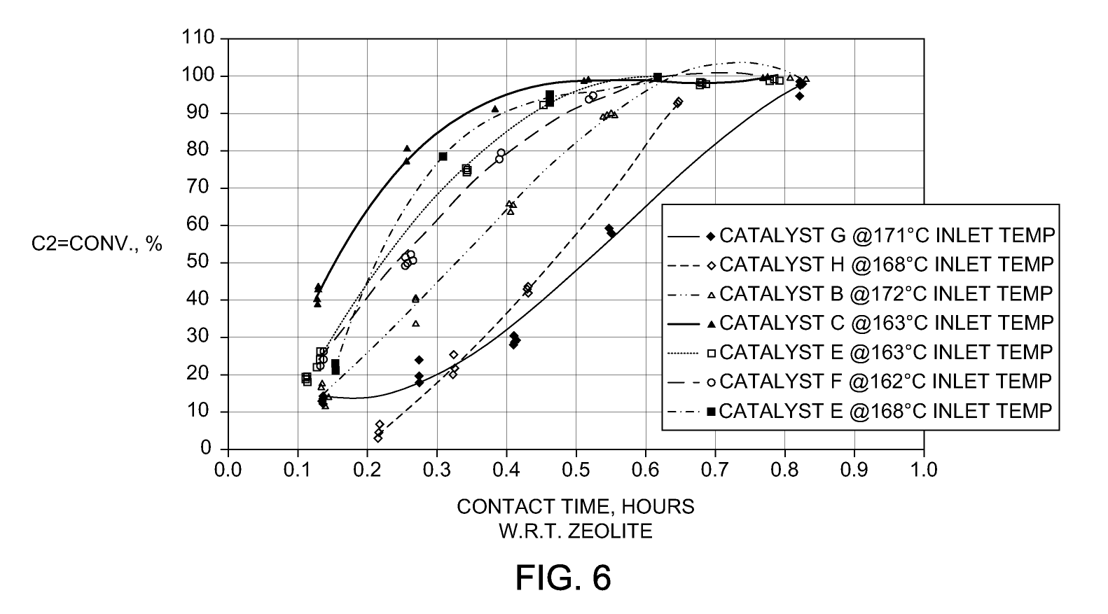

FIGS. 4 and 5 illustrate catalyst activity stability as measured by either the movement of temperature profiles, e.g., the movement of end of active zone, or by changes of unconverted propylene at approximately 40% into the catalyst bed as a function of WHSV. As shown in FIG. 6, Catalyst C made of UZM-8 zeolite showed little or no changes in the rates of movement of temperature profiles as WHSV doubled. As shown in FIG. 5, increasing WHSV with respect to Catalyst C also did not did not change the rate of propylene concentration increase with time at a sampling point approximately 40% into the catalyst bed.

The foregoing experimental results indicate that UZM-8 catalysts had better overall activity, selectivity and stability than MCM-22 catalysts in experiments simulating cumene production. These results indicated that UZM-8 catalysts, and in particular Catalyst C, could be used in a cumene alkylation process with a propylene WHSV of greater than 2.1 hr⁻¹, more particularly, between about 3.0 hr⁻¹ and about 4.0 hr⁻¹.

### Example 2

### Ethylbenzene Alkylation

Fresh benzene and fresh ethylene were introduced into a fixed bed reactor containing cylindrical extruded catalyst of 1/16″ diameter. A portion of the product effluent was recycled with an effluent to fresh feed ratio of 4.2 on a weight basis. A multiple point sampling device was installed so the product compositions including the unconverted ethylene were monitored along the catalyst bed by on-line gas chromatography. The temperature profiles along the catalyst bed were also monitored to provide another mean to gauge the catalyst activity. The catalyst was loaded at a constant volume and the performance was plotted against zeolite contact time to differentiate performance of different zeolite on a constant zeolite weight basis.

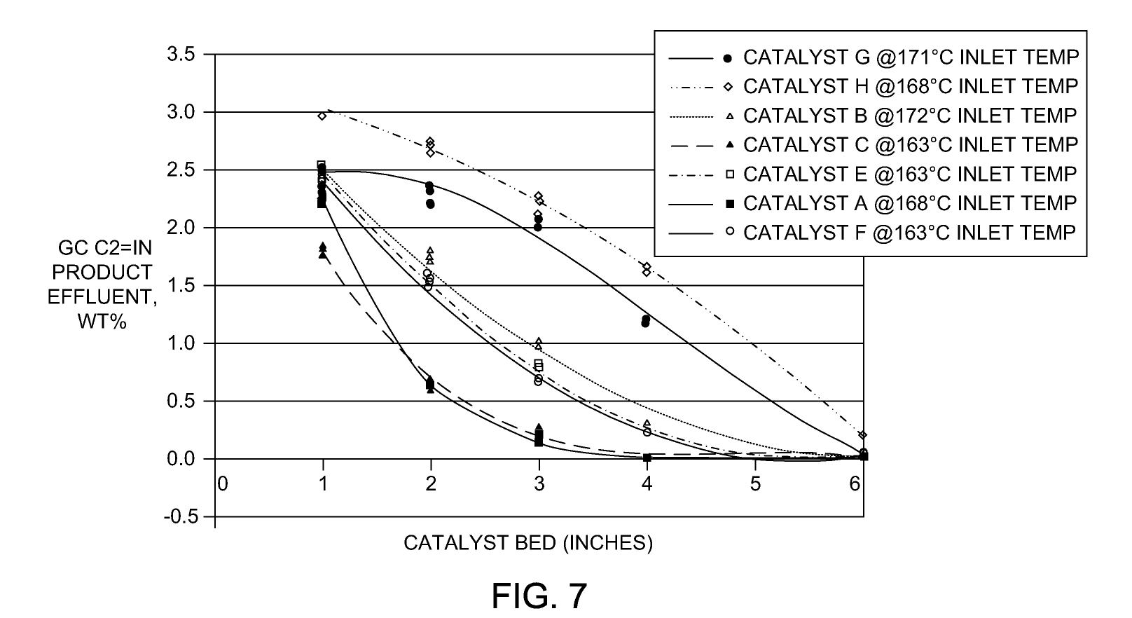

FIGS. 6 and 7 illustrate the results of Example 2. FIG. 6 is a chart illustrating percent conversion of ethylene versus contact time with the catalyst. FIG. 7 illustrates conversion rate as a percent of ethylene in the product effluent versus catalyst bed depth. The results indicate that UZM-8 Catalysts B, C, E and F showed high activity and nearly complete conversion at a lesser time period and catalyst bed depth. These results indicate that Catalyst C, for example, could be used in a commercial operation at an olefin WHSV of up to about 4 hr⁻¹ while still achieving complete olefin conversion. For higher value products, complete conversion (taking into account that it is difficult to achieve 100% conversion under laboratory conditions) occurred at about 0.5 hr. This indicates that Catalyst C could be used in a commercial operation at an olefin WHSV of between about 1.5 hr⁻¹ to about 3.0 hr⁻¹, more particularly, between about 1.5 hr⁻¹ to about 2.5 hr⁻¹, while still achieving complete olefin conversion. Moreover, UZM-8 Catalysts B, C, E and F all showed comparable activities to Catalyst A made of Beta zeolite and significantly higher activities than Catalysts H and I made of MCM-22 and -56 as per the prior art.

The foregoing experimental results indicate that UZM-8 catalysts had better overall activity than MCM-22 or MCM-56 catalysts in experiments simulating ethylbenzene production. These results indicate that UZM-8 catalysts, and in particular Catalyst C, could be used in an ethylbenzene alkylation process with an ethylene WHSV of greater than 1.5 hr⁻¹, more particularly, between about 2 hr⁻¹ and about 2.5 hr⁻¹.

1. A process for producing cumene comprising:
reacting a feed stream comprising benzene with an alkylating agent comprising propylene in a reaction zone in the presence of a UZM-8 or UZM-8HS catalyst to produce an effluent comprising cumene, wherein the length to diameter ratio for each catalyst bed in the reaction zone is between about 1.5 and 3, the alkylating agent WHSV is between about 2.1 hr⁻¹ and about 10.0 hr⁻¹, the reaction temperature is between about 60° and 160° C., and the molar ratio of benzene to propylene is between about 1 and about 3, and wherein the reaction results in complete olefin conversion with an end of active zone at approximately 40% into the catalyst bed.

2. The process of claim 1, wherein complete olefin conversion is defined to be greater than about 80% and the propylene WHSV is between about 4 hr⁻¹ and about 10 hr⁻¹.

3. The process of claim 2, wherein the alkylating agent comprises greater than about 50 wt % propylene.

4. The process of claim 1, wherein complete olefin conversion is defined to be greater than 95% and the propylene WHSV is between about 2.1 hr⁻¹ and about 4 hr⁻¹.

5. The process of claim 4, wherein the alkylating agent comprises at least about 70 wt % propylene.

6. The process of claim 1, wherein complete olefin conversion is defined to be greater than 95% and the propylene WHSV is between about 2.5 hr⁻¹ and about 3.5 hr⁻¹.

7. The process of claim 1 wherein the UZM-8 or UZM-8HS catalyst is derived from DEDMA or ETMA.

8. The process of claim 7 wherein the UZM-8 or UZM-8HS catalyst has a molar ratio of DEDMA:Al₂O₃ or ETMA:Al₂O₃ of between about 2 and about 6.

9. The process of claim 1 wherein the reaction zone includes a single reactor with multiple catalyst beds.

10. The process of claim 1 wherein the temperature at the reaction zone inlet is between about 110° C. and about 160° C.

11. A process for producing ethylbenzene comprising:
reacting a feed stream comprising benzene with an alkylating agent comprising ethylene in a reaction zone in the presence of a UZM-8 or UZM-8HS catalyst to produce an effluent comprising ethylbenzene, wherein the length to diameter ratio for each catalyst bed in the reaction zone is between about 1.5 and 3, the alkylating agent WHSV is between about 1.5 hr⁻¹ and about 4.0 hr⁻¹, the reaction temperature is between about 150° and 280° C., and the molar ratio of benzene to ethylene is between about 1 and about 4, and wherein the reaction results in complete olefin conversion with an end of active zone at approximately 40% into the catalyst bed.

12. The process of claim 11, wherein complete olefin conversion is defined to be greater than about 85% and the ethylene WHSV is between about 2.5 hr⁻¹ and about 4 hr⁻¹.

13. The process of claim 12, wherein the alkylating agent comprises greater than about 10 wt % ethylene.

14. The process of claim 11, wherein complete olefin conversion is defined to be greater than 95% and the ethylene WHSV is between about 1.5 hr⁻¹ and about 2.5 hr⁻¹.

15. The process of claim 14, wherein the alkylating agent comprises at least about 65 wt % ethylene.

16. The process of claim 11 wherein the UZM-8 or UZM-8HS catalyst is derived from DEDMA or ETMA.

17. The process of claim 16 wherein the UZM-8 or UZM-8HS catalyst has a molar ratio of DEDMA:Al₂O₃ or ETMA:Al₂O₃ of between about 2 and 6.

18. The process of claim 11 wherein the reaction zone includes a single reactor with multiple catalyst beds.
